(19) United States
(12) Patent Application Publication
(10) Pub. No.: US 2020/0046664 A1
(43) Pub. Date: Feb. 13, 2020

(54) METHOD FOR PREPARATION OF LIQUID ORAL COMPOSITION OF L-THYROXIN

(71) Applicant: FTF PHARMA PRIVATE LIMITED, Ahmedabad (IN)

(72) Inventors: Vijay PATEL, Ahmedabad (IN); Sandip MEHTA, Ahmedabad (IN); Manoj KALAVADIYA, Jamnagar (IN); Manish UMRETHIA, Ahmedabad (IN); Jayanta Kumar MANDAL, Ahmedabad (IN)

(21) Appl. No.: 16/340,790

(22) PCT Filed: Oct. 7, 2017
(86) PCT No.: PCT/IB2017/056204

§ 371 Date: Apr. 10, 2019

(30) Foreign Application Priority Data
Oct. 10, 2016 (IN) 201621034602

(51) Int. Cl. A61K 31/198; A61K 9/08

(52) U.S. Cl. CPC A61K 31/198; A61K 9/08

(57) ABSTRACT

The present invention discloses a processes for the preparation of a stable liquid pharmaceutical composition comprising levothyroxine or pharmaceutically acceptable salt thereof and one or more pharmaceutically acceptable excipients wherein the liquid composition of the present invention is used for oral administration. The present invention also discloses stable liquid compositions comprising levothyroxine or pharmaceutically acceptable salt thereof and one or more pharmaceutically acceptable excipients prepared according to the processes of the present invention.

### FIELD OF THE INVENTION

The present invention relates, in general, to the pharmaceutical field, and more precisely it relates to a process for preparing stable liquid pharmaceutical composition of levothyroxine (L-thyroxine) or its pharmaceutically acceptable salt thereof used for oral administration.

### BACKGROUND OF THE INVENTION

Levothyroxine, also known as L-thyroxine, synthetic T4, or 3,5,3′,5′-tetraiodo-L-thyronine, CAS number 51-48-9, is a synthetic form of thyroxine, used as a hormone substitute for patients with thyroid conditions, such as hypothyroidism, as well as conditions in which the thyroid gland becomes enlarged, causing swelling of the neck.

Thyroid hormones regulate multiple metabolic processes and play an essential role in normal growth and development, and normal maturation of the central nervous system and bone. Levothyroxine sodium was initially manufactured as synthetic T4 in 1958 and it was first introduced into the market as early as before 1962 without an approved NDA, apparently in the belief that it was not a new drug.

Levothyroxine sodium is very slightly soluble in water and slightly soluble in ethanol (96 percent). Levothyroxine sodium is described in the European Pharmacopoeia. The chemical designation of Levothyroxine sodium is Sodium (2S)-2-amino-3-[4-(4-hydroxy-3,5-diiodophenoxy)-3,5-diiodophenyl]propanoate. Its molecular formula is C₁₅H₁₀I₄NNaO₄, ×H₂O and its molecular weight is 799 (anhydrous substance). The structural formula is:

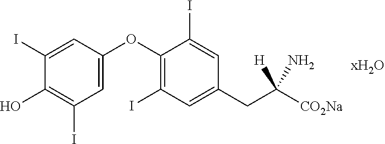

Orally administered levothyroxine sodium is used as replacement therapy in conditions characterized by diminished or absent thyroid function such as cretinism, myxedema, non-toxic goiter, or hypothyroidism (Food and Drug Administration 1997; Wertheimer and Santella 2005).

Levothyroxine Sodium Oral Solution is indicated for: hypothyroidism (congenital or acquired); diffuse nontoxicgoiter or Hashimoto's thyroiditis; thyroid carcinoma. The treatment of any thyroid disorder should be determined on an individual basis, taking account of clinical response, biochemical tests and regular monitoring. A pre-therapy ECG is valuable as changes induced by hypothyroidism may be confused with evidence of ischemia. If too rapid an increase of metabolism is produced (causing diarrhea, nervousness, rapid pulse, insomnia, tremors and sometimes anginal pain where there is latent myocardial ischemia), reduce the dose or withhold for 1-2 days and start again at a lower dose.

U.S. Pat. No. 5,225,204, incorporated herein by reference in its entirety, discloses a stabilized and uniform pharmaceutical formulation of Levothyroxine sodium comprising a complex of Levothyroxine sodium and a water soluble polyvinylpyrrolidone adsorbed on a cellulose compound in the form of a tablet, powder or capsule. Further, U.S. Pat. No. 5,225,204 also discloses a stabilized and uniform pharmaceutical formulation of levothyroxine sodium comprising a complex of levothyroxine sodium and a block copolymer of ethylene oxide and propylene oxide adsorbed on a cellulose compound in the form of a tablet, powder or capsule. Further, U.S. Pat. No. 5,225,204 also discloses a stabilized pharmaceutical formulation of levothyroxine sodium comprising levothyroxine sodium substantially uniformly adsorbed on a cellulose compound in the form of a tablet, powder or capsule.

U.S. Pat. No. 5,635,209, incorporated herein by reference in its entirety, discloses a method of making levothyroxine sodium medication by combining together: (a) levothyroxine sodium mixed with a carrier; (b) potassium iodide mixed with a carrier; (c) a disintegrant, and d. a lubricant. Further, U.S. Pat. No. 5,635,209 also discloses a method of making levothyroxine sodium medication by combining together: (a) a mixture of levothyroxine sodium mixed with microcrystalline cellulose, said mixture comprised of 1.05% levothyroxine sodium and 98.95% microcrystalline cellulose; (b) a second mixture of potassium iodide mixed with microcrystalline cellulose, said second mixture comprised of potassium iodide ranging from 0.1% to 0.7% and microcrystalline cellulose ranging from 99.9% to 99.3%; (c) croscarmellose sodium; (d) magnesium stearate, and (e) colored dye. Further, U.S. Pat. No. 5,635,209 also discloses a medication consisting of the combination of levothyroxine sodium with potassium iodide.

U.S. Pat. No. 6,491,946, incorporated herein by reference in its entirety, discloses a pharmaceutical composition comprising levothyroxine sodium, potassium iodide, microcrystalline cellulose and hydroxypropylmethylcellulose or gelatine or both hydroxypropylmethylcellulose and gelatine, which is essentially free of antioxidants. Further, U.S. Pat. No. 6,491,946 also discloses a process for the preparation of a pharmaceutical composition comprising levothyroxine sodium, potassium iodide, microcrystalline cellulose and a binding agent, which is essentially free of antioxidants, said process comprising spraying an aqueous hydroxypropylmethylcellulose and/or gelatine solution comprising levothyroxine sodium and potassium iodide which are present in suspended form in said solution onto microcrystalline cellulose in a fluidized bed granulation, admixing a disintegrating agent and a lubricant and compressing the resultant mixture to form tablets.

U.S. Pat. No. 6,555,581, incorporated herein by reference in its entirety, discloses a stable, solid, immediate release pharmaceutical composition for oral administration to treat a thyroid disorder, said composition comprising: (a) about 0.00005 wt % to about 5 wt % of a levothyroxine salt; (b) at least about 50 wt % of a β-form microcrystalline cellulose particles, said β-form microcrystalline cellulose particles having generally flat needle-shapes, a bulk density in a range of from about 0.10 g/cm³ to about 0.23 g/cm³, and a conductivity of less than about 200 μS/cm; and (c) about 0.5 wt % to about 30 wt % disintegrating agent; wherein (1) at least about 90 wt % of the levothyroxine salt in the composition dissolves in an aqueous solution in less than about 5 minutes, (2) potency loss for said levothyroxine salt is no more than about 0.3% per month for a period of at least about 18 months, and (3) said composition is essentially sugar-free.

U.S. Pat. No. 6,646,007, incorporated herein by reference in its entirety, discloses a process for the production of a pharmaceutical preparation, comprising spraying levothyroxine sodium and optionally liothyronine sodium, in suspended form in aqueous gelatin solution, onto a filler(s) in a fluidized bed granulation, admixing a disintegrant and lubricant and compressing the mixture to give tablets.

U.S. Pat. No. 7,101,569, incorporated herein by reference in its entirety, discloses a method of administering a levothyroxine pharmaceutical composition to a patient, comprising placing an immediate release levothyroxine pharmaceutical tablet that loses less than about 0.7% potency per month for up to 18 months in an aqueous medium, dispersing the levothyroxine composition in the aqueous medium for less than ten minutes, and administering the aqueous medium to the patient.

U.S. Pat. No. 7,955,621, incorporated herein by reference in its entirety, discloses a pharmaceutical formulation in unit dose form which is a “50 μg tablet” of active ingredient comprising: 0.0425-0.0575 mg levothyroxine sodium, 50-60 mg microcrystalline cellulose which has a mean particle size of less than 125 μm, 12-17 mg pregelatinised starch which is produced by subjecting moistened starch to mechanical pressure in order to rupture some or all of its starch granules and subsequent drying, 2-3 mg talc, 1-2 mg colloidal anhydrous silica, and 0.5-1.0 mg magnesium stearate. Further, U.S. Pat. No. 7,955,621 also discloses a pharmaceutical formulation in unit dose form which is a “100 μg tablet” of active ingredient comprising: 0.085-0.115 mg levothyroxine sodium, 100-120 mg microcrystalline cellulose which has a mean particle size of less than 125 μm, 24-34 mg pregelatinised starch which is produced by subjecting moistened starch to mechanical pressure in order to rupture some or all of its starch granules and subsequent drying, 4-6 mg talc, 2-4 mg colloidal anhydrous silica, and 1-2 mg magnesium stearate.

U.S. Pat. No. 8,008,349, incorporated herein by reference in its entirety, discloses a pharmaceutical preparation comprising active compound, 5 mg gelatin as a binder and fillers and which is free of organic solvent, wherein the active compound is levo-thyroxine sodium, and which preparation is in tablet form. Further, U.S. Pat. No. 8,008,349 also discloses a pharmaceutical preparation comprising levo-thyroxine sodium, 5 mg gelatin as a binder and fillers which is free of organic solvent and which is in solid form. Further, U.S. Pat. No. 8,008,349 also discloses a pharmaceutical preparation comprising active compound, 5 mg gelatin as a binder and fillers and which is free of organic solvent wherein the active compound consists essentially of levo-thyroxine sodium and liothyronine sodium, and which preparation is in solid form.

U.S. Pat. No. 8,293,272, incorporated herein by reference in its entirety, discloses a solid pharmaceutical preparation comprising water-soluble salts of levothyroxine as an active ingredient, wherein water activity of said pharmaceutical preparation is adjusted to values of below 0.4 and above 0.1, measured at a room temperature of 25° C.

U.S. Pat. No. 8,333,192, incorporated herein by reference in its entirety, discloses a device comprising an inhaler suitable for administration of a stable dry powder blend, said device containing a blend comprising a) levothyroxine sodium hydrate, b) lactose particles, comprising lactose H₂O, gelatine and starch maize, c) sodium starch glycolate, d) magnesium stearate, and e) talc silicified, comprising talc purified and colloidal silicon dioxide; and wherein the dry powder comprises levothyroxine sodium in an amount 4 to 0.02 mg per 100 mg of the dry powder.

U.S. Pat. No. 9,006,289, incorporated herein by reference in its entirety, discloses a composition, comprising: about 100 or about 200 micrograms of levothyroxine sodium; a phosphate buffer; and from 2 to 4 milligrams of mannitol, where the composition is a lyophilized solid.

U.S. Pat. No. 9,050,307, incorporated herein by reference in its entirety, discloses a method for the preparation of an oral levothyroxine composition, the method comprising the steps of: a) providing a salt of levothyroxine, b) mixing levothyroxine with an aqueous solvent, c) adjusting the pH to a pH of at least 8 to yield a basic aqueous solvent, d) dissolving the levothyroxine in the basic aqueous solvent to yield a levothyroxine solution, and e) lowering the pH of the clear levothyroxine solution to between 5-6. Further, U.S. Pat. No. 9,050,307 also discloses an oral Levothyroxine composition prepared according to the process described therein, wherein the composition comprises a sodium levothyroxine concentration of approximately 25 μg in 5 ml, approximately 50 μg in 5 ml or approximately 100 μg in 5 ml, wherein the composition comprises sodium levothyroxine, glycerol, water and a preservative.

U.S. Pat. No. 9,168,238, incorporated herein by reference in its entirety, discloses a lyophilized solid composition, comprising: about 100 micrograms of levothyroxine sodium; a buffer; and between 2 and 4 milligrams of mannitol.

U.S. Pat. No. 9,168,239, incorporated herein by reference in its entirety, discloses a lyophilized solid composition, comprising: between 100 and 500 micrograms of a salt of levothyroxine; a buffer; and between 2 and 4 milligrams of mannitol.

U.S. Pat. No. 9,271,951, incorporated herein by reference in its entirety, discloses a pharmaceutical composition comprising thyroxine, acacia, and about 0.001% wt % to about 0.5% wt % of an antioxidant selected from propyl gallate, butylatedhydroxyanisol, and butylatedhydroxytoluene, and wherein the composition comprises from about 0.1% wt % to about 10% wt % acacia.

U.S. Pat. No. 9,345,772, incorporated herein by reference in its entirety, discloses a pharmaceutical solution comprising: from 0.001% w/v to 0.01% w/v of a levothyroxine; from 75% w/w to 95% of glycerol; from 0.01% w/w to 1.5% w/w of an ethylenediaminetetraacetic acid (EDTA); and an amount of water sufficient to adjust the pharmaceutical solution to 100% w/w, and wherein the solution is storage stable.

Oral solutions of levothyroxine are particularly suitable for use in children and in the elderly who may have difficulty to swallow tablets. Unfortunately, solutions of levothyroxine are less stable compared to tablets during storage. Also, levothyroxine solutions may comprise relatively high amounts of liothyronine, which is believed to be the source of side-effects in certain patients. Aqueous levothyroxine solutions are prone to decomposition compared to the solid forms. The big advantage of the solution is the uniformity of dosage units in comparison to solid dosage forms (tablets). The tablets, usually due to the very low levothyroxine content (0.04% up to 0.5% w/w), have problems of content uniformity during the production process and many times the actual content that the patient receives with tablet therapy, is not 100% but could range from 85% up to 120% and this creates serious problems on patient treatment. In contrast, it is much easier to obtain a homogeneous solution.

### OBJECTS OF THE INVENTION

It is therefore one of the principal object of the present invention to provide processes for preparing a stable liquid composition comprising levothyroxine (L-thyroxine) or pharmaceutically acceptable salt thereof wherein the stable liquid composition of levothyroxine is suitable for oral administration.

A yet another object of the present invention is to provide stable liquid compositions comprising levothyroxine (L-thyroxine) or pharmaceutically acceptable salt thereof prepared according to the processes of the invention wherein the stable liquid composition is suitable for oral administration.

### DETAILED DESCRIPTION OF THE INVENTION

Oral administration of drug is considered to be the most important and convenient method for maximum effectiveness of the drug molecules. Liquid dosage form is the most common and widely accepted dosage form for having advantages such as faster absorption than solid, palatable, better choice for children and old age patients, more flexibility in achieving the proper dosage of medication and provides ease for the patients having difficulty in swallowing other oral dosage forms.

Oral liquid dosage form provides one of the suitable ways for the formulating a dosage form with high stability and high solubility. Solution is a homogenous liquid preparation that contains one or more dissolved medicaments.

The process of the present invention offers an opportunity for the preparation of liquid dosage forms comprising levothyroxine having poor water solubility. The liquid compositions of present invention comprise active pharmaceutical ingredient levothyroxine or pharmaceutically acceptable salts thereof, one or more solvents, one or more preservatives, one or more pH adjusting agents and one or more vehicles or combinations thereof.

The present invention provides method of preparing stable pharmaceutical compositions of levothyroxine or pharmaceutically acceptable salts thereof as active ingredient used for oral administration wherein the stable pharmaceutical composition is liquid composition.

According to one of the preferred embodiments of the present invention, the process for the preparation of oral liquid compositions of levothyroxine or its pharmaceutically acceptable salts comprises following steps.

Process-1
(i) Mix levothyroxine or pharmaceutically acceptable salt thereof in one or more water-miscible organic solvent with or without adding water;
(ii) Adjust pH of the mixture obtained in step (i) to alkaline pH, preferably between 8.0 and 10.0 using one or more suitable pH adjusting agents;
(iii) Add one or more pharmaceutically acceptable preservative into the mixture obtained in step (ii) followed by pH adjustment to acidic pH, preferably between 5.0 and 6.0;
(iv) Add purified water into the mixture obtained in step (iii) to adjust final required volume and mix well.

According to one of the further preferred embodiments of the present invention, the process for the preparation of oral liquid compositions of levothyroxine or its pharmaceutically acceptable salts comprises following steps.

Process-2
(i) Dissolve one or more pharmaceutically acceptable preservative in the mixture of pharmaceutically acceptable solvents, preferably an aqueous solvent and a non-aqueous solvent;
(ii) Adjust the pH of the mixture obtained in step (i) to about neutral to alkaline pH, between 7.0 and 8.0, preferably between 7.1 and 7.5 using one or more suitable pH adjusting agents;
(iii) Disperse levothyroxine or pharmaceutically acceptable salt thereof in pharmaceutically acceptable solvent, preferably a non-aqueous solvent with or without adding water;
(iv) Add Step (iii) into Step (ii) and mix till levothyroxine or pharmaceutically acceptable salt thereof gets dissolved;
(v) Adjust the pH of the mixture obtained in Step (iv) to acidic pH, preferably between 5.0 and 6.0 using one or more suitable pH adjusting agents;
(vi) Add pharmaceutically acceptable solvent, preferably purified water to the mixture obtained in step (v).

Those who are reasonably skilled in the art can understand that some variations in the above described processes for the preparation of liquid compositions of the present invention can be adopted without affecting the quality and characteristics of the resulting product which are well within the scope of the skilled artisan. One can change sequences of one or more steps in the above mentioned processes for the purposes of suitability and convenience without affecting the quality and characteristics of the resulting product. Such other processes are also well within the scope of the present invention.

Surprisingly, above mentioned processes result in a levothyroxine compositions which are more stable during storage. The obtained compositions also comprise less amounts of liothyronine impurity. Also, the preparation is relatively fast; in spite that levothyroxine is dissolved in, (i) a non-aqueous solvent with or without adding water and (ii) dissolving levothyroxine at about pH 7.0, preferably between pH 7.1 and 7.5.

The term “about” in the context of the present invention denotes an interval of accuracy that the person skilled in the art will understand to still ensure the technical effect of the feature in question. The term typically indicates deviation from the indicated numerical value of ±10%, and preferably ±5%.

The provided levothyroxine salt and other ingredients are all of pharmaceutical quality. The pH is determined and monitored, preferably using a calibrated electronic pH meter based on electrode potential. In order to determine the pH during adjusting the pH, the pH should be adjusted by adding small amounts of pH adjusting agents to the mixture while stirring, and allowing to homogenize and stabilize the measured pH before proceeding to further adjust the pH. The end pH 5.0-6.0 is suitable for storage as well as for administering the levothyroxine solution to a patient.

As levothyroxine may show degradation under the influence of UV and blue light, the process can be performed in the dark or in dark glass comprising a UV-filter. Best results can be obtained when in step (ii) of the Process-1 as well as Process-2 the pH is adjusted between 8.0 and 11.0, preferably between 8.0 and 10.0. Surprisingly, the compositions of the present invention are also stable when in step (ii) of the Process-2, the pH is adjusted between 7 and 8, preferably between 7.1 and 7.5.

It is preferred if the adjustment of the pH is done by adding alkalizing agents, preferably a base. It is preferred if the base is added as an aqueous solution. Suitable bases according to the present invention comprise without limitation Potassium Bicarbonate, Potassium Citrate, Potassium Hydroxide, Sodium Carbonate, Ammonium carbonate, Calcium Hydroxide, Ammonia Solution, Sodium Hydroxide, Sodium dibasic phosphate, Sodium Borate, Monoethanolamine, Sodium Citrate, Diethanolamine, Triethanolamine and Sodium Bicarbonate.

Preferably, adjustment of the pH in step (iii) of Process-1 and in step (v) of Process-2 can be done using one or more acidifying agents. Suitable acidifying agents according to the present invention comprise without limitation Hydrochloric acid, Nitric acid, Sulfuric acid, Propionic acid, Fumaric acid, Lactic acid, Phosphoric acid, Malic acid, Lauric Acid, Tartaric Acid, Acetic Acid, Maleic Acid, Citric acid and Sorbic Acid. In a preferred embodiment, the carboxylic acid is citric acid, which was well tolerated, compatible with levothyroxine and gave good results.

It is preferred if the levothyroxine or pharmaceutically acceptable salt thereof is dissolved in non-aqueous solvents, preferably water-miscible organic solvent with or without adding water. It is also preferred if the levothyroxine or pharmaceutically acceptable salt thereof is dissolved in an aqueous solvent. Aqueous solvent as used herein can be selected without limitation from the group comprising of purified water, hydro-alcoholic solvents, and polyhydric alcohols. Aqueous solvent may also be referred as a mixture of water and a water-miscible organic solvent or solubilizer. Water-miscible organic solvents improves the speed of dissolving and gives a stable solution. Preferably the water-miscible organic solvent according to the present invention comprises glycol. Suitable organic solvents or solubilizers according to the present invention comprise without limitation Acetone, Alcohol, Benzyl Alcohol, Benzyl Benzoate, Butylene Glycol, Dibutyl Phthalate, Diethyl Phthalate, Dimethyl Phthalate, Dimethyl Sulfoxide, Dimethylacetamide, Glycofurol, Glycerin, Isopropyl Alcohol, Isopropyl Myristate, Isopropyl Palmitate, Polyethylene Glycol, Propylene Carbonate, Pyrrolidone, Triacetin, Triethyl Citrate and Triolein.

One or more preservatives can also be added to the composition of the present invention. This yields an increased stability during the storage. Suitable preservatives according to the present invention comprise without limitation Bronopol, Imidurea, Potassium Sorbate, Phenoxyethanol, Phenylmercuric Acetate, Butylparaben, Benzyl Alcohol, Phenylmercuric Borate, Chlorocresol, Benzethonium Chloride, Phenylethyl Alcohol, Benzalkonium Chloride, Methylparaben, Hexetidine, Chlorobutanol, Ethylparaben, Propylparaben, Sodium Benzoate, Potassium Benzoate, Sorbic Acid, Cresol, Propylparaben Sodium, Cetylpyridinium Chloride, Phenylmercuric Nitrate, Chloroxylenol, Propionic Acid, Phenol, Thimerosal, Sulfur Dioxide, Boric Acid, Edetic Acid, Sodium Propionate, Calcium Chloride, Sodium Acetate, Sodium Sulfite, Benzoic Acid, Monothioglycerol, Cetrimide, Calcium Acetate, Butylene Glycol, Sodium Metabisulfite, Alcohol, Propyl Gallate, Potassium Metabisulfite, Sodium Lactate, Chlorhexidine, Calcium Lactate, Pentetic Acid, Glycerin, Propylene Glycol Alginate, Sodium Borate, Magnesium Trisilicate, Isopropyl Alcohol, Dimethyl Ether, Propylene Glycol, Butylated Hydroxyanisole, Pyrrolidone, Lactic Acid, Sodium Lauryl Sulfate and Dimethyl Sulfoxide. Preferably, the preservative is sodium methylparahydroxybenzoate, which showed a good compatibility with levothyroxine.

The invention further provides a liquid oral levothyroxine composition obtainable using the methods of the present invention. The composition comprising levothyroxine or its pharmaceutically acceptable salt, preferably a sodium salt has concentration of approximately 25 μg in 5 ml, approximately 50 μg in 5 ml or approximately 100 μg in 5 ml. In a preferred embodiment, the composition comprises sodium levothyroxine, glycerine, purified water and a preservative.

In a preferred embodiment, the oral levothyroxine sodium composition is packed in a unit dose system selected from the group consisting of ampoules, sachets, vials, blister packs, tubes, of stick packs, wherein the unit dose is arranged to deliver separate doses of levothyroxine from 25 μg up to 300 μg per single dose.

### BEST MODE OF CARRYING OUT THE INVENTION EXAMPLES

The processes according to the present invention for the preparation of pharmaceutical composition comprising levothyroxine or its pharmaceutically acceptable salt is explained in more detail with reference to the following examples. These examples are provided by way of illustration of the present invention only and should not be construed as to limit the scope of the claims in any manner.

The process can be performed using regular manufacturing vessel and equipment except direct contact of the light/sunlight. Levothyroxine may degrade under the influence of light/sunlight and therefore the process may be performed avoiding direct contact from the light/sunlight.

### Example-1: Process for Preparing Levothyroxine Sodium Liquid Compositions

The basic process includes following steps.
(i) After weighing accurately levothyroxine or pharmaceutically acceptable salt thereof was dispersed in the sufficient quantity of Glycerin with or without water to initiate the process.
(ii) Aqueous solution of sodium hydroxide was added in the dispersion of step (i) to adjust the pH between 8.0 and 10.0 under stirring and stirred well until clear solution is obtained.
(iii) Sodium methyl parahydroxybenzoate was added to the solution obtained in step (ii) and stirred well to obtain homogenous clear solution.
(iv) Citric acid monohydrate was added in sufficient quantity to adjust the pH of the solution obtained in step (iii) between 5.3 and 5.6.
(v) The final volume of the solution obtained in step (iv) was made up with the required quantity of purified water. The pH of the final composition was kept between 5.0 and 6.0.
(vi) The final solution was filtered over a polypropylene 10p filter, and filled in light-protective containers, such as amber type III glass 100 ml bottles sealed with child resistant, tamper evident screw caps.

### Example-2: Process for Preparing Levothyroxine Sodium Liquid Compositions

The basic process includes following steps.
(i) After weighing accurately, Glycerin and Purified water are mixed to initiate the process.
(ii) Sodium methyl parahydroxybenzoate is dissolved in the mixture of Glycerin and Water obtained in step (i).
(iii) Citric acid monohydrate was added in the mixture obtained in step (ii) in sufficient quantity to adjust the pH between 7.1 and 7.3.
(iv) Accurately weighed levothyroxine sodium was dispersed in Glycerin. (v) The dispersion obtained in step (iv) was added to the mixture obtained in step (iii) under stirring and stirred until a clear solution was obtained. (vi) Citric acid monohydrate was added in the solution obtained in step (v) in sufficient quantity to adjust the pH between 5.3 and 5.6.
(vii) The final volume of the solution obtained in step (vi) was made up with the required quantity of purified water. The pH of the final composition was kept between 5.0 and 6.0.
(viii) The final solution was filtered over a polypropylene 10μ filter, and filled in light-protective containers, such as amber type III glass 100 ml bottles sealed with child resistant, tamper evident screw caps.

Preferably, the doses of levothyroxine are packed in dose units or monodose delivery systems of the levothyroxine solution. Such systems comprise without limitation sealed vessels holding dosed units mentioned above. The vessels are made for instance of PVC or PVDC or composite materials comprising plastic materials reinforced with aluminum and/or glass layers for a better protection from light. These vessels are appropriate for pharmaceutical use and have volumes from 1 upto 10 ml capable to deliver doses from 25 μg up to 300 μg of levothyroxine sodium. The vessels may have the form of an ampoule, sachet, vial, blister pack, tube, or a stick pack made from plastic or glass.

Oral solutions of different concentrations may be obtained using the methods described above. The method above may be scaled up or down using techniques known in the art to obtain compositions having different quantities and/or concentrations. Compositions containing alternate excipients equivalent to those exemplified in above paragraphs can be prepared using processes of the invention with variations in the sequences of one or more steps. Such other similar compositions and process variations are also within the ambit of the present invention.

### Example-3: Comparative Stability Study of the Levothyroxine Sodium Liquid Composition Prepared According to the Process of the Present Invention

The levothyroxine sodium liquid pharmaceutical compositions prepared according to the processes of the present invention exhibit unexpected stability profile when tested after two months kept under the conditions 40° ° C.±2° C./75%±5% RH. The liquid compositions prepared according to the processes of the present invention possess comparatively less amount of liothyronine and highest degree of purity.

Table-1 shows comparative stability studies between the levothyroxine sodium compositions of the present invention, commercially available levothyroxine sodium formulation (Evotrox®) and prior known levothyroxine sodium compositions (Pharma-Data) as disclosed and provided in U.S. Pat. No. 9,050,307.

TABLE 1
Comparative Study of Levothyroxine sodium composition 100 mcg/5 ml

| | Evotrox ® | | Pharma-Data | | | |
|---|---|---|---|---|---|---|
| | EVX011 | | 100 mcg/5 ml | | Composition of the | |
| | | 40° C. | | 40° C. | present invention | |
| Test | | 2 | | 2 | | 40° C. |
| Parameters | Initial | Months | Initial | Months | Initial | 2 Months |
| Appearance | Almost Clear | | Clear Solution | | Clear Solution | |
| pH | 5.6 | | 5.5 | | 5.8 | |
| Liothyronine | 0.81% | 4.8% | 0.19% | 0.77% | ND | 0.54% |
| Any other | 1.4% | 3.4% | 0.12% | 0.8% | ND | 0.46% |
| individual | | | | | | |
| impurity | | | | | | |
| Total | 3.4% | 5.4% | 0.28% | 1.1% | ND | 0.46% |
| impurities^{#} | | | | | | |

^{#}withoutliothyronine;
ND = Not Detected

From above stability data it can be concluded that the levothyroxine sodium liquid compositions prepared according to the processes of the present invention unexpectedly show improved and better stability profile as compared to the commercially available levothyroxine sodium compositions (Evotrox®) as well as prior known levothyroxine sodium compositions (Pharma-Data). The processes described herein above unexpectedly provides stable levothyroxine sodium compositions even when the levothyroxine sodium is dissolved at pH between 7.1 and 7.5 instead of prior disclosed and preferred pH 10.0. Dissolution of levothyroxine sodium in a water-miscible organic solvent alone without adding water instead of mixture of water and a water-miscible organic solvent does not diminish quality of the final product of the present invention and unexpectedly provides improved and better stability profile even after two months. The liquid compositions prepared according to the processes of the present invention have comparatively low amount of liothyronine as compared to the commercially available product (Evotrox®) as well as prior known levothyroxine sodium compositions (Pharma-Data as described in the U.S. Pat. No. 9,050,307 which is incorporated herein by reference in its entirety). Thus, the present invention provides a fast and convenient scalable process for manufacturing stable liquid compositions of levothyroxine sodium.

It should be understood that various changes and modifications to the presently preferred embodiments and examples described herein will be apparent to those skilled in the art. Such changes and modifications can be made without departing from the spirit and scope of the present subject matter and without diminishing its intended advantages. It is therefore intended that such changes and modifications be covered by the appended claims.

1. A process for the preparation of an oral liquid pharmaceutical composition comprising levothyroxine or pharmaceutically acceptable salt thereof, the process comprising steps of:
(i) adding levothyroxine or pharmaceutically acceptable salt thereof in one or more non-aqueous solvent with or without adding an aqueous solvent;
(ii) adjusting pH of a mixture obtained in step (i) between 8.0 and 10.0 using one or more pH adjusting agents;
(iii) adding one or more pharmaceutically acceptable preservatives into a mixture obtained in step (ii) followed by pH adjustment between 5.0 and 6.0 using one or more pH adjusting agents; and
(iv) adding one or more aqueous solvents into a mixture obtained in step (iii) to make up final required volume and followed by mixing.

2. A process for the preparation of an oral liquid pharmaceutical composition comprising levothyroxine or pharmaceutically acceptable salt thereof, the process comprising steps of:
(i) dissolving one or more pharmaceutically acceptable preservatives in a mixture of one or more aqueous solvents and one or more non-aqueous solvents;
(ii) adjusting pH of a mixture obtained in step (i) between 7.0 and 8.0 using one or more pH adjusting agents;
(iii) dispersing levothyroxine or pharmaceutically acceptable salt thereof in a non-aqueous solvent without adding an aqueous solvent;
(iv) adding mixture of step (iii) into step (ii) and mixing till levothyroxine or pharmaceutically acceptable salt thereof gets dissolved;
(v) adjusting pH of a mixture obtained in step (iv) between 5.0 and 6.0 using one or more pH adjusting agents; and
(vi) adding one or more aqueous solvent to a mixture obtained in step (v) to make up final required volume.

3. The process according to claim 1, wherein one or more pH adjusting agents used to adjust pH between 8.0 and 10.0 are selected from the group consisting of Potassium Bicarbonate, Potassium Citrate, Potassium Hydroxide, Sodium Carbonate, Ammonium carbonate, Calcium Hydroxide, Ammonia Solution, Sodium Hydroxide, Sodium dibasic phosphate, Sodium Borate, Monoethanolamine, Sodium Citrate, Diethanolamine, Triethanolamine, Sodium Bicarbonate, and combination thereof.

4. The process according to claim 1, wherein one or more non-aqueous solvents are selected from the group consisting of Acetone, Alcohol, Benzyl Alcohol, Benzyl Benzoate, Butylene Glycol, Dibutyl Phthalate, Diethyl Phthalate, Dimethyl Phthalate, Dimethyl Sulfoxide, Dimethylacetamide, Glycofurol, Glycerin, Isopropyl Alcohol, Isopropyl Myristate, Isopropyl Palmitate, Polyethylene Glycol, Propylene Carbonate, Pyrrolidone, Triacetin, Triethyl Citrate, Triolein, and combination thereof.

5. The process according to claim 1, wherein one or more aqueous solvents are selected from the group consisting of purified water, hydro-alcoholic solvents, polyhydric alcohols, and combinations thereof.

6. The process according to claim 1, wherein one or more pharmaceutically acceptable preservatives are selected from the group consisting of Bronopol, Imidurea, Potassium Sorbate, Phenoxyethanol, Phenylmercuric Acetate, Butylparaben, Benzyl Alcohol, Phenylmercuric Borate, Chlorocresol, Benzethonium Chloride, Phenylethyl Alcohol, Benzalkonium Chloride, Methylparaben, Hexetidine, Chlorobutanol, Ethylparaben, Propylparaben, Sodium Benzoate, Potassium Benzoate, Sorbic Acid, Cresol, Propylparaben Sodium, Cetylpyridinium Chloride, Phenylmercuric Nitrate, Chloroxylenol, Propionic Acid, Phenol, Thimerosal, Sulfur Dioxide, Boric Acid, Edetic Acid, Sodium Propionate, Calcium Chloride, Sodium Acetate, Sodium Sulfite, Benzoic Acid, Monothioglycerol, Cetrimide, Calcium Acetate, Butylene Glycol, Sodium Metabisulfite, Alcohol, Propyl Gallate, Potassium Metabisulfite, Sodium Lactate, Chlorhexidine, Calcium Lactate, Pentetic Acid, Glycerin, Propylene Glycol Alginate, Sodium Borate, Magnesium Trisilicate, Isopropyl Alcohol, Dimethyl Ether, Propylene Glycol, Butylated Hydroxyanisole, Pyrrolidone, Lactic Acid, Sodium Lauryl Sulfate, Dimethyl Sulfoxide, and combinations thereof.

7. The process according to claim 1, wherein one or more pH adjusting agents used to adjust pH between 5.0 and 8.0 are selected from the group consisting of Hydrochloric acid, Nitric acid, Sulfuric acid, Propionic acid, Fumaric acid, Lactic acid, Phosphoric acid, Malic acid, Laurie Acid, Tartaric Acid, Acetic Acid, Maleic Acid, Citric acid, Sorbic Acid, and combinations thereof.

8. An oral liquid composition comprising levothyroxine or pharmaceutically acceptable salt thereof and one or more pharmaceutically acceptable excipients prepared using a process comprising:
(i) adding levothyroxine or pharmaceutically acceptable salt thereof in one or more non-aqueous solvent with or without adding an aqueous solvent;
(ii) adjusting pH of a mixture obtained in step (i) between 8.0 and 10.0 using one or more pH adjusting agents;
(iii) adding one or more pharmaceutically acceptable preservatives into a mixture obtained in step (ii) followed by pH adjustment between 5.0 and 6.0 using one or more pH adjusting agents; and
(iv) adding one or more aqueous solvents into a mixture obtained in step (iii) to make up final required volume and followed by mixing.

9. The oral liquid composition according to claim 8, wherein one or more pharmaceutically acceptable excipients are selected from the group consisting of solvents, preservatives, pH adjusting agents, and combination thereof.

10. The oral liquid composition according to claim 8, wherein the composition comprises a sodium levothyroxine concentration of about 25 μg to about 100 μg in 5 mL, and wherein the composition comprises sodium levothyroxine, glycerin, purified water and a preservative.

11. The process according to claim 2, wherein one or more pH adjusting agents are selected from the group consisting of Potassium Bicarbonate, Potassium Citrate, Potassium Hydroxide, Sodium Carbonate, Ammonium carbonate, Calcium Hydroxide, Ammonia Solution, Sodium Hydroxide, Sodium dibasic phosphate, Sodium Borate, Monoethanolamine, Sodium Citrate, Diethanolamine, Triethanolamine, Sodium Bicarbonate, and combination thereof.

12. The process according to claim 2, wherein one or more non-aqueous solvents are selected from the group consisting of Acetone, Alcohol, Benzyl Alcohol, Benzyl Benzoate, Butylene Glycol, Dibutyl Phthalate, Diethyl Phthalate, Dimethyl Phthalate, Dimethyl Sulfoxide, Dimethylacetamide, Glycofurol, Glycerin, Isopropyl Alcohol, Isopropyl Myristate, Isopropyl Palmitate, Polyethylene Glycol, Propylene Carbonate, Pyrrolidone, Triacetin, Triethyl Citrate, Triolein, and combination thereof.

13. The process according to claim 2, wherein one or more aqueous solvents are selected from the group consisting of purified water, hydro-alcoholic solvents, polyhydric alcohols, and combinations thereof.

14. The process according to claim 2, wherein one or more pharmaceutically acceptable preservatives are selected from the group consisting of Bronopol, Imidurea, Potassium Sorbate, Phenoxyethanol, Phenylmercuric Acetate, Butylparaben, Benzyl Alcohol, Phenylmercuric Borate, Chlorocresol, Benzethonium Chloride, Phenylethyl Alcohol, Benzalkonium Chloride, Methylparaben, Hexetidine, Chlorobutanol, Ethylparaben, Propylparaben, Sodium Benzoate, Potassium Benzoate, Sorbic Acid, Cresol, Propylparaben Sodium, Cetylpyridinium Chloride, Phenylmercuric Nitrate, Chloroxylenol, Propionic Acid, Phenol, Thimerosal, Sulfur Dioxide, Boric Acid, Edetic Acid, Sodium Propionate, Calcium Chloride, Sodium Acetate, Sodium Sulfite, Benzoic Acid, Monothioglycerol, Cetrimide, Calcium Acetate, Butylene Glycol, Sodium Metabisulfite, Alcohol, Propyl Gallate, Potassium Metabisulfite, Sodium Lactate, Chlorhexidine, Calcium Lactate, Pentetic Acid, Glycerin, Propylene Glycol Alginate, Sodium Borate, Magnesium Trisilicate, Isopropyl Alcohol, Dimethyl Ether, Propylene Glycol, Butylated Hydroxyanisole, Pyrrolidone, Lactic Acid, Sodium Lauryl Sulfate, Dimethyl Sulfoxide, and combinations thereof.

15. The process according to claim 2, wherein one or more pH adjusting agents used to adjust pH between 5.0 and 8.0 are selected from the group consisting of Hydrochloric acid, Nitric acid, Sulfuric acid, Propionic acid, Fumaric acid, Lactic acid, Phosphoric acid, Malic acid, Laurie Acid, Tartaric Acid, Acetic Acid, Maleic Acid, Citric acid, Sorbic Acid, and combinations thereof.

16. An oral liquid composition comprising levothyroxine or pharmaceutically acceptable salt thereof and one or more pharmaceutically acceptable excipients prepared using a process comprising:
(i) dissolving one or more pharmaceutically acceptable preservatives in a mixture of one or more aqueous solvents and one or more non-aqueous solvents;
(ii) adjusting pH of a mixture obtained in step (i) between 7.0 and 8.0 using one or more pH adjusting agents;
(iii) dispersing levothyroxine or pharmaceutically acceptable salt thereof in a non-aqueous solvent without adding an aqueous solvent;
(iv) adding mixture of step (iii) into step (ii) and mixing till levothyroxine or pharmaceutically acceptable salt thereof gets dissolved;
(v) adjusting pH of a mixture obtained in step (iv) between 5.0 and 6.0 using one or more pH adjusting agents; and
(vi) adding one or more aqueous solvent to a mixture obtained in step (v) to make up final required volume.

17. The oral liquid composition according to claim 16, wherein one or more pharmaceutically acceptable excipients are selected from the group comprising of solvents, preservatives, pH adjusting agents, and combination thereof.

18. The oral liquid composition according to claim 8, wherein the composition comprises a sodium levothyroxine concentration of about 25 μg to about 100 μg in 5 mL, and wherein the composition comprises sodium levothyroxine, glycerin, purified water and a preservative.
